(19) United States
(12) Patent Application Publication
(10) Pub. No.: US 2022/0080416 A1
(43) Pub. Date: Mar. 17, 2022

(54) MICROFLUIDIC DEVICE AND METHOD FOR RAPID HIGH THROUGHPUT IDENTIFICATION OF MICROORGANISMS

(71) Applicant: CITROGENE INC., San Jose, CA (US)

(72) Inventors: S. Abbas Hosseini, Los Altos, CA (US); Mahsa Karbaschi, San Jose, CA (US); Amir Tork, San Jose, CA (US)

(73) Assignee: CITROGENE INC.

(21) Appl. No.: 17/476,729

(22) Filed: Sep. 16, 2021

Related U.S. Application Data
(60) Provisional application No. 63/079,929, filed on Sep. 17, 2020.

(51) Int. Cl. B01L 3/00; G01N 33/569; B01L 7/00

(52) U.S. Cl. CPC B01L 3/502715; G01N 33/56983; B01L 7/52; B01L 2300/0867; C12Q 1/686; B01L 2300/0803; B01L 2200/16; B01L 2400/0409; B01L 2300/0663

(57) ABSTRACT

An apparatus and method are disclosed for detecting the presence of a microorganism within sampling device. The sampling device has a plurality of reaction chambers each having a reactive reagent for reacting with the microorganism to indicate the presence of the microorganism within the reaction chamber. A grabber holding the sampling device and a motion stage connected to the grabber moves the sampling device in a plane. A detector detects each of the plurality of reaction chambers for detecting the presence of the microorganism within the reaction chamber.

### CROSS-REFERENCE TO RELATED APPLICATIONS

This application claims benefit of U.S. Patent Provisional Application No. 63/079,929 filed Sep. 17, 2020. All subject matter set forth in Provisional Application No. 63/079,929 is hereby incorporated by reference into the present application as if fully set forth herein.

### BACKGROUND OF THE INVENTION

### Field of the Invention

The present invention is related to the field of medical diagnostics and more particularly to a microfluidic device designed to manage all of the experimental steps to detect microorganisms, such as Covid-19 virus or other potential infectious pathogens in a single sample body.

### Description of the Related Art

With the Covid-19 pandemic, a reliable, inexpensive, easy to use diagnostic device is needed. Currently, there are attempts to do the Covid-19 tests in major ports of entries, and some companies in Europe and the United States do tests on their employee before entering into the workplace. In several countries, tests are being conducted in major affected regions. Typically, these tests are conducted using PCR (Polymerase Chain Reaction) methodology which is highly effective and a precise way of diagnosis. However, PCR equipment is expensive, requires a clinical lab with highly trained professionals to perform the tests and it take about two (2) hours to complete each test.

Currently with the Covid-19 pandemic, there are a number of territories that are requiring travelers to self-quarantine upon arrival for two weeks. England and Canada have also put in place similar self-quarantine requirements. Germany requires mandatory testing of each traveler upon arrival, and only allow travelers with negative covid-19 test results within the last 72-96 hours prior to arrival to the destination country. Although the test is free of charge for arriving travelers, the departing passengers are required to pay for the test to avoid the quarantine restrictions by their destination country. Presently the costs are, around $59 for a test that you have results in 12 hours or $165 for a test that you have results in 6 hours. For these tests, the passenger's mucosa samples are collected by nasopharyngeal swabs and then sent out to a clinic for PCR test.

Currently mass spectrometry testing systems are deployed in almost all airports. These systems are used to detect explosive and radioactive materials on travelers and are there to protect the public. These systems can detect very small amounts of radioactive or explosive materials if the passenger has been in close contact to these materials. Presently, these mass spectrometers are run by security personnel with minimum training.

With the current Covid-19 pandemic, and other possible highly infectious diseases in the future, there is a rapid need for a machine that can be run by a security personnel with minimum training to test for infectious diseases. The test needs to be fast, precise, and most of all cost-effective. Also, because of the large number of travelers a high-throughput testing system is needed to test multiple travelers or employees at one time, so batch processing is important to avoid costly delays. In addition, this testing system can be deployed in schools and colleges to test students before entering the classrooms. In any of the scenarios listed above if there is any indication of the disease in the traveler, employee or student can be isolated and thus minimize the spread of the virus to other travelers, employees, students, or family members. The present Covid-19 pandemic has had significant negative economic impacts as well as being detrimental to our education of our youth and it has had major impact on public mental health.

Another major issue is the similarities between influenza and Covid-19 symptoms, our healthcare systems should be able to distinguish between these illnesses to avoid public panic and extra hospital costs. The present applicant has been working on a compact infectious diseases diagnostic device for analytical biochemical systems incorporating a lab-on-chip microfluidic sampling devices since early 2019. The original idea was to make a device to examine citrus trees for Huanglongbing disease (HLB or Yellow Dragon Disease). Trees affected with HLB have stunted growth, beer multiple off-season flowers (most of which fall off), and produce small irregularly shaped fruit with thick, pale peel that remains green ant the bottom and tastes very bitter, thus effecting the profitability of the trees. Once a tree is infected, the tree must be removed as well as 10 trees around the infected tree, this is extremely costly for the farmers. Technique deployed was to use the genetic fingerprint of the HLB bacteria for the detection. A machine is under development to enable farmers with minimum training to precisely test for the HILB in the field.

After Covid-19 started to become a world-wide problem, the present applicant decided to use the generic design of the microfluidic sampling device to develop a fast and reliable method for the detection of COVID-19 and other new microorganisms. The sampling device design is generic as such it can be used for any other infectious disease diagnostics in epidemic or pandemic situations such as Malaria, Fhola, Zika, Anthrax (biological weapons), etc.

The microfluidic disk of the present invention could be used as a platform for the high-throughput diagnosis of infectious diseases by a variety of molecular biology-based methodologies, such as Loop Mediated Isothermal Amplification (LAMP), Reverse Transcription LAMP (RT-LAMP), CRISPR-Cas diagnosis, PCR, RT-PCR and the like.

The foregoing has outlined some of the more pertinent objects of the present invention. These objects should be construed as being merely illustrative of some of the more prominent features and applications of the invention. Many other beneficial results can be obtained by modifying the invention within the scope of the invention. Accordingly, other objects in a full understanding of the invention may be had by referring to the summary of the invention, the detailed description describing the preferred embodiment in addition to the scope of the invention defined by the claims taken in conjunction with the accompanying drawings.

### SUMMARY OF THE INVENTION

The present invention is defined by the appended claims with specific embodiments being shown in the attached drawings. To summarize, the invention relates to an apparatus and a method for the rapid identification of a microorganism (here more specific Covid-19) within a microfluidic sampling device. The microfluidic sampling device has a plurality of reaction chambers each having a reactive agent for reacting with the microorganism. The reaction with the microorganism indicates the presence of a microorganism in the reaction chamber.

In one embodiment, the invention relates to an apparatus for identifying microorganism within a sampling device. The sampling device has a plurality of reaction chambers each having a reactive agent for reacting with the microorganism to indicate the presence of the microorganism in the reaction chamber. The apparatus comprises a grabber for holding the sampling device. A motion stage is connected to the grabber for moving the sampling device in a plane. A detector detects each of the plurality of reaction chambers for detecting the presence of a microorganism in the reaction chamber.

In a more specific embodiment, the invention relates to an apparatus for detecting the Covid-19 virus within sampling device. The sampling device has a plurality of reaction chambers each having a reactive agent for reacting with Covid-19 virus to indicate the presence of Covid-19 virus in the reaction chamber. The apparatus comprises a grabber for holding the sampling device. A motion stage is connected to the grabber for moving the sampling device in a plane. A detector detecte each of the plurality of reaction chambers for detecting the presence of a Covid-19 virus in the reaction chamber.

In another specific embodiment, the invention relates to a microfluidic sampling device for identifying the presence of Covid-19 from a potential patient sample. The microfluidic sampling device comprises a sample body having an inlet for receiving the reactive reagents and the patient sample. A reservoir receives the clinical sample and a reactive reagent from the inlet. A channel transfers the patient sample and the reactive agent from the reservoir to a reaction chamber. The mixer channel may include a torturous path for mixing the patient sample and the reactive agent prior to entering the reaction chamber. Alternatively, the patient sample and the reactive agent may be mixed to each other by shaking prior to entering the reaction chamber. The reaction chamber is transparent for enabling the detection of the presence of Covid-19 within the reaction chamber. The bottom and top layer of reaction chamber is coated with a transparent electrode to heat the mixture at the specific controlled temperature.

In still another specific embodiment, the invention relates to a microfluidic sampling device for identifying the presence of Covid-19 from a patient sample. The microfluidic sampling device comprises a rotary disk having a plurality of inlets located on and inner region of the rotary disk for receiving potential patient samples. A plurality of reservoir receives the clinical sample and the reactive agent from the plurality of inlets. A plurality of reaction chambers is located on the outer periphery of the rotary disk. A channel transfers the patient samples and the reactive agent from each of the plurality of reservoirs to each of the plurality of reaction chambers. Each of the channels may include a torturous path for mixing each of the patient samples with the reactive agents prior to entering each of the reaction chambers. Alternatively, the patient sample and the reactive agent may be mixed to each other by shaking prior to entering the reaction chamber. The reaction chambers are transparent for enabling detection of the presence of Covid-19 within the reaction chamber.

The invention is also incorporated into a method for rapid identification of Covid-19 in a potential patient sample comprising the steps of introducing the patient sample into a reservoir. A reagent is introduced into the reservoir. The patient sample and the reagent are moved through a microchannel to mix the patient sample with the reagent. The mixed patient sample and reagent are moved to a reaction chamber. The patient sample and reagent are heated within the reaction chamber. The presence of Covid-19 is detected within the reaction chamber. Preferably, the step of moving the patient sample and the reagent includes moving the patient sample and reagent by centrifugal force.

The invention is also incorporated into a method for rapid identification of the presence of infectious microorganisms from a potential patient sample comprises the steps of introducing the patient sample into a reservoir. A reagent is introduced into the reservoir. The patient sample and the reagent are moved through a microchannel to mix the patient sample with the reagent. The mixed patient sample and reagent are moved to a reaction chamber through microchannel which helps further mixing. The patient sample and reagent are heated within the reaction chamber. The presence of the infectious microorganisms is detected within the reaction chamber.

In a more specific embodiment of the invention, the microfluidic sampling device comprises of an inlet for receiving a reactive agent and a sample. A reservoir holds the injected materials. The sample and reactive agent are transferred through capillary microchannels that act as mixer before transferring to the reaction chamber. A reaction of the reactive reagent with the microorganism indicates the presence of a microorganism in the reaction chamber. Preferably, the sample body is transparent and fabricated from glass or thermo plastics. Each of the plurality of reaction chambers is sequentially illuminated for detecting the presence of a microorganism in the reaction chamber. Three reaction chambers may be assigned to work as test controls. The present invention offers different platforms, to serve the diagnostic application on different bodily fluids such as blood, urine, saliva, semen, mucosa and the like.

The specific design platform for the detection of Covid-19 in nasopharyngeal samples is explained hereinafter. In one example, the microfluidic sampling device is formed in the shape of a microfluidic rotary disk. However, the specific design of the microfluidic sampling device may take various shapes and forms depending upon the desired tests to be performed.

Firstly, a microfluidic rotary disk is inserted into the machine for initializations of the assay. Specific reagents which are prepared and loaded in a cartridge are injected into the allocated inlets on the rotary disk. The nasopharyngeal specimen samples which are collected by nasal swabs are inserted in the buffer tube and mixed with a cell containing a lysis solution. The samples are injected into the disk.

In one example, 30 patient samples can be loaded into each disk at a time. The rotary disk has 63 reaction chambers, 3 of them will be used as relevant test controls to check for the accuracy of the tests at different levels. The other 60 channels will be used for 30 unknown individual cases, two reaction chambers for each sample.

One reaction chamber is allocated for the detection of one human housekeeping gene such as human rActin which serves as a control to test the accuracy of the extraction of genetic material from the clinical samples. The second reaction chamber is allocated for the evaluation of the presence or absence of the pathogen genomic fingerprint. In the case of Covid-19, the presence of the virus nucleocapsid gene (N-gene) or envelope gene (E-gene) are investigated. In the alternative, the presence of N-gene and Orfl-gene (open reading frame) are investigated. The platform structure is very generic which could have widespread applications by including specific cartridges assigned for other types of infectious disease such as Malaria, Ebola, Zika, Anthrax, and the like.

In order to run LAMP assays as an example on the proposed platform, the biomaterial reaction including the viral or bacterial specific primers (set of 4-6 forward and reverse primers) is primarily injected into an inlet of the disk followed by the injection of the extracted genetic materials into the same inlet. The mixture of the genetic material and the reaction is shacked for further mixing, then transferred towards the microchannels and the final reservoir by controlled centrifugal force during rotation. Passing through the microchannels provides a vigorous mixture of the multiple injected materials. When the reaction mix reaches the final reservoir, the final reservoir is heated using the patterned transparent electrode fixed on top and bottom layer of microfluidic sampling device to one constant temperature of 65° C. to let the amplification process start for the duration of 20-30 minutes. Heating the samples can be done using a small oven inside the device as well. The amplification process produces signals which can be detected through simple spectrometer detector.

If the infectious disease is caused by RNA viruses (e.g., retroviruses), the LAMP reaction assay could be coupled with a reverse transcriptase. This could produce a cDNA molecule prior to the LAMP reaction. In another word, a one-step amplification of the RNA molecules could be run on this disk.

The amount of double strand DNA produced through LAMP is considerably higher than the PCR-based amplification methods. The LAMP process eliminates the need for the expensive PCR machines to provide precise thermal cycles, gel electrophoresis equipment and trans-illuminators. The LAMP process is a more suitable method of detection in the point of cares with low resources.

The spectrometer detector can be programmed to either read the final signal at the end of the reaction or read the signal in multiple intervals to provide a real-time reading of the signal. If the viral or bacterial load in a patient sample is high, the availability of an option for a real time detection of the signal could reduce the diagnosis time to less than 30 minutes as the signal is produced earlier.

The real-time detection of the signals adds the possibility of running qPCR reactions on these rotary disks. This requires a thermal controller device to be adjustable for different temperatures, to perform like a thermal cycler which could read the fluorescent signal in intervals.

The rotary disk and its bio-processor of the present invention is also compatible with the diagnostic assays which are run with more than one individual reaction material in multiple steps. For example, diagnostic CRISPR-Cas 12/13a methodology could be run on this rotary disk. In this process, the amplification reaction mix, and genetic material are both injected through the inlet of the rotary disk. The material is then moved to the first reservoir by centrifugal force and is kept there for an adjustable time period under specific temperature (61-65° C.) for the genetic material to be amplified. At the end of this step, a second reaction material (CRISPR-Cas proteins and random reporter molecules) is added to the rotary disk through the inlet and then both solutions are piloted through microchannels for mixing prior to reaching the second reservoir. The reagents in the second reservoir then get heated to 37° C. for 10-30 minutes and the fluorescent signal is detected and measured in adjustable time points to monitor the signal. The system then automatically normalizes the signals against the signals produced from the non-transcribed controls and provides the results automatically.

The present invention is based on the development of a new glass based microfluidic device. This is possible to change the design in a day and have the sampling device ready to function the next day by cutting the glass in desired shape and bond it on the bottom and top by two other glass layers. All biochemical steps including the injection of different materials, local mixing, centrifugation, pumping the liquid and valving, degassing, thermalizing, and final real time detection are all done in a single microfluidic disk.

Another major advantage is to use minimum and optimum volume of biomaterials and samples to do many tests at the same time. The present invention may use 20 μL of biomaterials and 5 μL of clinical samples, but is not limited to this ratio. The read out can be accomplished after 20-25 minute with accurate results. The overall device is portable and light weight with low power consumption. The size of microfluidic sampling device is similar to a compact disk (CD) with cartage carrier, injector, temperature controller, light source, and spectrometer.

The present microfluidic sampling device is cost effective due to multiple analyses in each run. The disk has CD size structure and while empty, it can be stored from −70° C. to +70° C. without any needs for refrigeration or specific location to store. Therefore, the disk can be stored in the room temperature without needing any refrigeration. The disk can be preloaded with reactive materials. In this case it needs to be kept at −20° C. and be used within the recommended shelf life period. The device disk is recyclable, cost effective, and has less harm to the environment. The present invention is user friendly and requires minimal technical training and can be used at point of cares, hospital, airports (borders), schools, work environments, entertainment centers, and the like.

The foregoing has outlined rather broadly the more pertinent and important features of the present invention in order that the detailed description that follows may be better understood so that the present contribution to the art can be more fully appreciated. Additional features of the invention will be described hereinafter which form the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a full understanding of the nature and objects of the invention, reference should be made to the following detailed description taken in connection with the accompanying drawings in which:

FIG. 1 is a side view of an apparatus for rapid identification of microorganisms in the present invention;

FIG. 2A illustrates a base layer of a microfluidic sampling device shown as a rotary disk;

FIG. 2B illustrates a main of patterned main layer of the rotary disk wherein a reaction will take place;

FIG. 2C is a magnified view of the FIG. 2B;

FIG. 2D illustrates a cover layer of the rotary disk;

FIG. 3A-B illustrate a cartridge top view and isomorphic view respectively;

FIG. 4A illustrates a first portion of a molecular biology-based methodologies; and

FIG. 4B illustrates a second portion of a molecular biology-based methodologies;

FIG. 5A presents the detected spectrum for the positive, negative controls and water;

FIG. 5B presents the detected spectrum for the positive, negative controls and water,

FIG. 5C presents the detected spectrum for the positive, negative controls and water;

FIG. 6 presents patterned transparent electrode for an accurate local heating of the disk sample; and

FIG. 7 presents the isomorphic view of the sample tube holder.

### DETAILED DISCUSSION

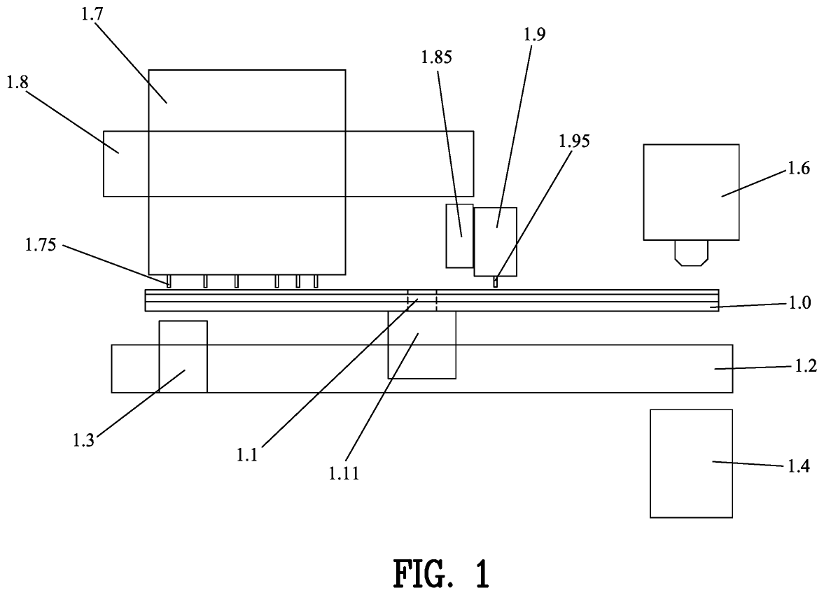

FIG. 1 illustrates an apparatus for rapid identification of a microorganism such as a bacteria, fungi and viruses. The apparatus is suitable for use with LAMP, RT-LAMP CRISPR-CAS 12/13 diagnostics method, PCR, and RT-PCR. The apparatus receives a microfluidic sampling device 1.0. As will be described in greater detail hereinafter, the microfluidic sampling device 1.0 receives a sample for analysis. The microfluidic sampling device 1.0 is interchangeable and disposable for rapid identification of a microorganism. The microfluidic sampling device 1.0 is able to diagnose the infectious diseases in sample such as blood, mucosa, saliva, semen, urine. However, the microfluidic sampling device 1.0 can be designed for virus, bacteria or fungi detection as well. It always looks for the genetic fingerprint of the pathogen under detection.

In this example, the microfluidic sampling device 1.0 is shown as a disk 1.0 having an approximate size of a compact disk (CD). However, the specific design of the micro fluidic sampling device may take various shapes and forms depending upon the desired tests to be performed. The apparatus comprises a grabber 1.1 for holding the rotary disk 1.0. The grabber 1.1 includes a motor 1.11 mounted on the base 1.2 for rotating the rotary disk 1.0.

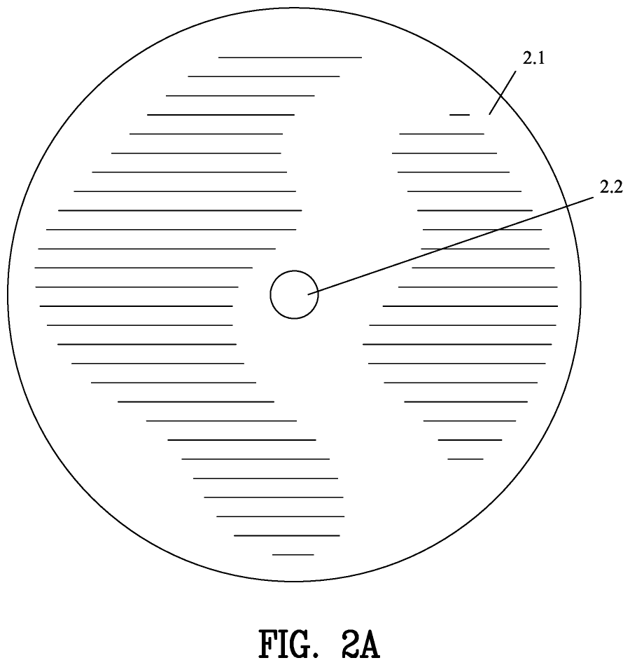

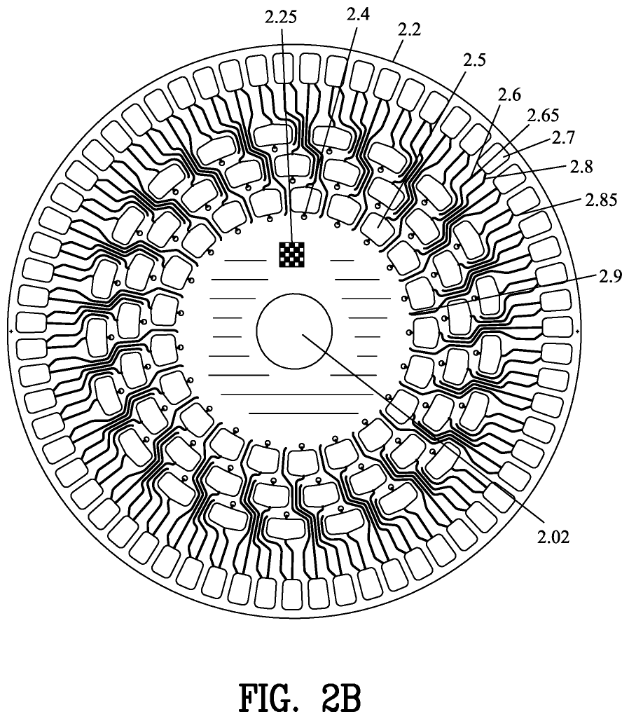

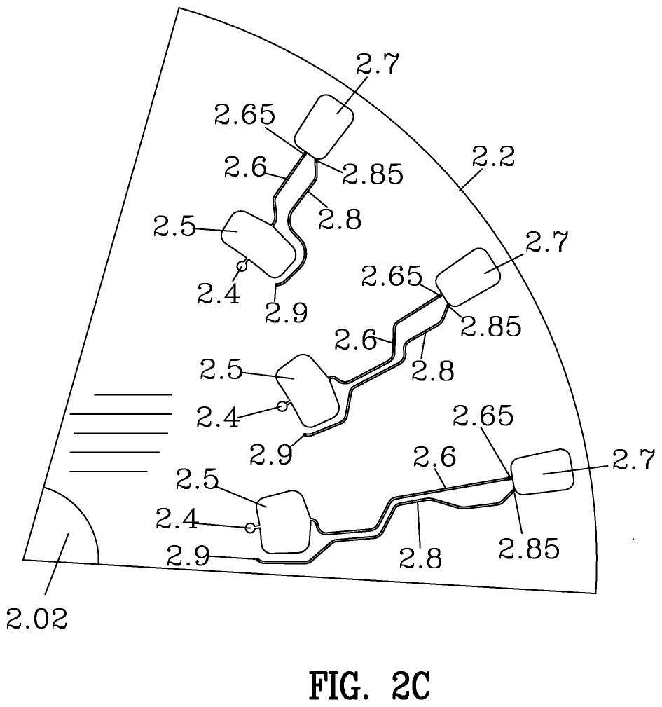

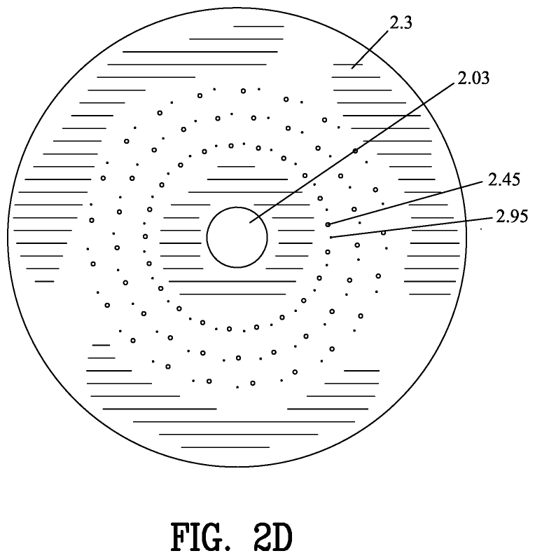

As will be described in greater detail hereinafter, a light source 1.4 is located under the rotary disk 1.0 and aligned to reaction chambers 2.7 in the rotary disk 1.0. A spectrometer detector 1.6 measures the light passing through and/or emitted from selective reaction chambers 2.9 of the rotary disk 1.0. Cartridge 1.7 is located on top of the disk 1.0 to inject biomaterials and clinical samples to the disk 1.0 through needle 1.75. The cartridge 1.7 is inserted on a linear rail 1.8 as such cartridge can move towards the center of the disk or reverse. The combination of this motion and rotation of the disk 1.0 make sures that all six needles in the cartridge can access the inlet 2.4 (FIG. 2C). In another embodiment the disk is preloaded with biomaterials and only patient samples are inserted into the disk. Also sample tube 1.9 is located on a rotation stage 1.85 (cross section is shown) and needle 1.95 well aligned with the inlet 2.4 of the disk 1.0. By rotation of the stage 1.85, 30 sample tubes inject in 60 inlets. Each patient sample injects in two inlets for the internal control and the gene of interest in the pathogen (N and E genes for COVID-19). There is a heating system 1.3 that can heat the disk up to 65° C. This heater can be set at a constant temperature or perform like a thermal cycler.

FIGS. 2A-D are enlarged views of a microfluidic sampling device 1.0 incorporated into a rotary disk 1.0. The rotary disk 1.0 comprises a base layer 2.1 formed from transparent material such as glass or any other material with preferred thickness of 100-1100 m as a structural base of the rotatable disk 1.0. The base layer 2.1 stabilizes the remaining thinner layers of the rotary disk 1.0. A hole 2.2 is defined in rotary disk 1.0 for enabling the grabber 1.1 to hold and to rotate the rotary disk 1.0.

FIG. 2B is the main layer of the rotary disk 1.0. It has a concentric central hole forming a part of the hole 2.02 of the rotary disk 1.0. The main layer 2.2 is formed from transparent material such as glass or any other materials. All the rotary disks 1.0 have specific assigned identification number such as a barcode 2.25 that is printed on the main layer 2.2.

FIG. 2C is a magnified view of the disk 1.0 presenting three levels of microfluidic which is called leaf. All these three leaf design perform same function. An inlet 2.4 is defined in the main layer 2.2 to inject the reactive agent and sample. The reaction agent will be injected to the reservoir 2.5. The volume of this reservoir is intentionally designed to be few micrometers more than the volume of total injected material.

When the sample is injected through inlet 2.4, the sample is pushed to the middle of reactive reagent in the reservoir 2.5. The mixing of the sample and reagents start here due to intrinsic liquid diffusion and shaking. After loading the materials (reactive reagents and the samples) the rotary disk 1.0 begins to shake for few seconds then rotate at 2000 rpm for 10 seconds. The intrinsic centrifugal force accurately regulates the motion of the mixture during rotation. The microchannels 2.6 define a resistive path to provide extra resistance to the flow of liquid therethrough. Microchannel 2.6 carries mixed liquids outward towards reaction chamber 2.7. This extra resistance promotes mixing of the sample and reactive agents. There is no full access from microchannel 2.6 to the reaction chamber 2.7. A siphon valve 2.65 is installed in between. This valve ensures the liquid will trap in the reaction chamber 2.7 and will not come back to the microchannel 2.6 when disk stops the rotation. An outlet microchannel 2.8 through exit hole of 2.9 is designed to vent all possible trapped air during rotation or biochemical reaction. Similarly another siphon valve 2.85 is installed in the path of reaction chamber 2.7 and vent channel 2.8 to prevent skipping of the liquid from reaction chamber.

The excess of the air in the microchannels 2.6 and 2.8 as well as the chambers 2.5, and 2.7 push the liquid back towards the inlet 2.4 when rotary disk 1.0 stops the rotation. The outlet microchannel 2.8 evacuates any trapped air through siphon channel 2.85. However, the location of the exit of the outlet microchannel 2.8 is very important. Due to Coriolis force that happens in the rotary disk 1.0, air tends to move towards 5 O'clock position. If the outlet microchannel 2.8 is located at 12 O'clock position, the liquid goes through the outlet microchannel 2.8 and the air will be trapped inside the reaction chamber 2.7 creating air bubbles. This means losing some portion of the liquid resulting in a lower signal. The outlet microchannel 2.8 is intentionally made towards the center of the disk as such liquid itself cannot be discharged from the rotary disk 1.0 while rotating. When the rotary disk 1.0 stops, then sample and reactive agents are mixed and placed in the reaction chamber 2.7.

The existence of a siphon valve structure 2.65 at the inlet of the reaction chamber 2.7 and a siphon valve 2.85 at the outlet of the reaction chamber 2.7 creates resistance against capillary effect and traps the liquid in the reaction chamber 2.7. This type of valving is referred to as siphon valving. Siphon valving is very effective and simple and performs with no additional valving cost.

FIG. 2D illustrates a cover layer 2.3. The cover layer 2.3 has a concentric central hole of 2.03 for grabber 1.1. It has inlet holes 2.45 in alignment with the inlets 2.4 in the main layer 2.2 to inject the liquids as well as outlet holes 2.95 in alignment with the air outlet holes 2.9 in the main layer 2.2 to evacuate air.

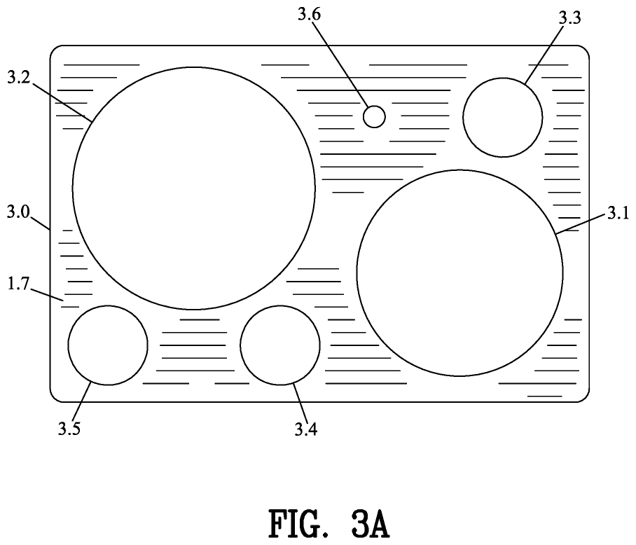

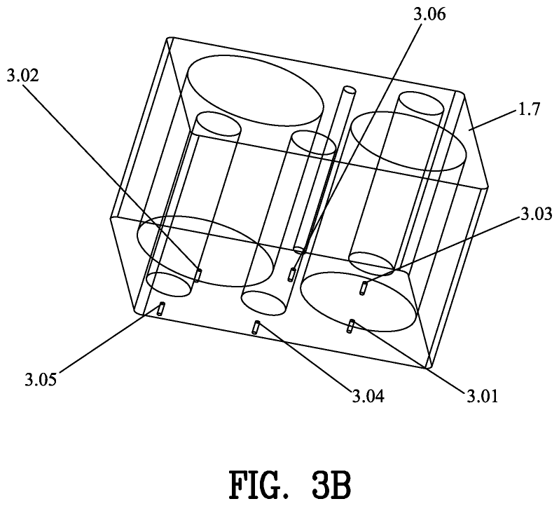

FIG. 3A illustrates top view of the cartridge 3.0 for the present invention. In this example, the content of the cartridge 1.7 as shown in FIG. 1 or 3.0 as shown in FIG. 3A will be explained with specific reference to Covid-19, but it should be understood that the content of the cartridge 3.0 may be adapted for other types of testing.

Preferably, cartridge 3.0 is a molded plastic that has six different containers. Container 3.1 holds the ddH₂O. Container 3.2 holds the common biomaterial such as enzymes. Container 3.3 carries primers for housekeeping gene, for example, rActin primer is used as the internal control. Container 3.4 contains primer for N gene and E gene of Covid-19 and container 3.5 contains primer for O gene of Covid-19. Container 3.6 holds the synthesized Covid-19 RNA.

The cartridge 3.0 can vary in size. For example, a 100 disks load to run 3000 tests requires 45 mL of common biomaterial, 4.25 mL of N gene primer, 4.25 mL of E gene primer, 4.25 mL of primers for housekeeping gene, and 300 μL of synthesized Covid-19 RNA and finally 32.5 mL of ddH₂O. FIG. 3B is the isomorphic view of the cartridge. It presents the six needles 3.01 to 3.06 for the injection. In another embodiment of present invention all the material explained for cartridge 3.0 are preloaded in the disk and use only prepare and loads the patient sample.

Loop-mediated isothermal amplification (LAMP) method relies on the auto cycling strand displacement of DNA molecules [1-4]. The assay is based on using 2-3 primer pairs (4-6 primers) which specifically recognize 6-8 different areas of target DNA. A strand-displacing DNA polymerase initiates synthesis at a constant temperature with greater efficiency. To improve the amplification process, there are also 2 specially designed primers to create loop structures. The DNA products of LAMP assay include several repeats of the short target sequences which is linked together through single-stranded loop sequences. Although LAMP products are not applicable for further manipulations, LAMP products are very suitable for the detection of pathogens due to the extensive amplification.

The following publications investigate the process of amplifying nucleic acids. K Nagamine, T. H., T Notomi, Method of synthesizing single-stranded nucleic acid. 2000, Eiken Chemical Co Ltd. Notomi, T., et al., Loop-mediated isothermal amplification of DNA. Nucleic Acids Res, 2000. 28(12): p. E63. TSUGUNORI, H. T. N., Method of synthesizing nucleic acid. EIKEN CHEMICAL. Tsugunori Notomi, K. N., Method of amplifying nucleic acid by using double-stranded nucleic acid as template. 2003, Eiken Chemical Co Ltd.

### Primers

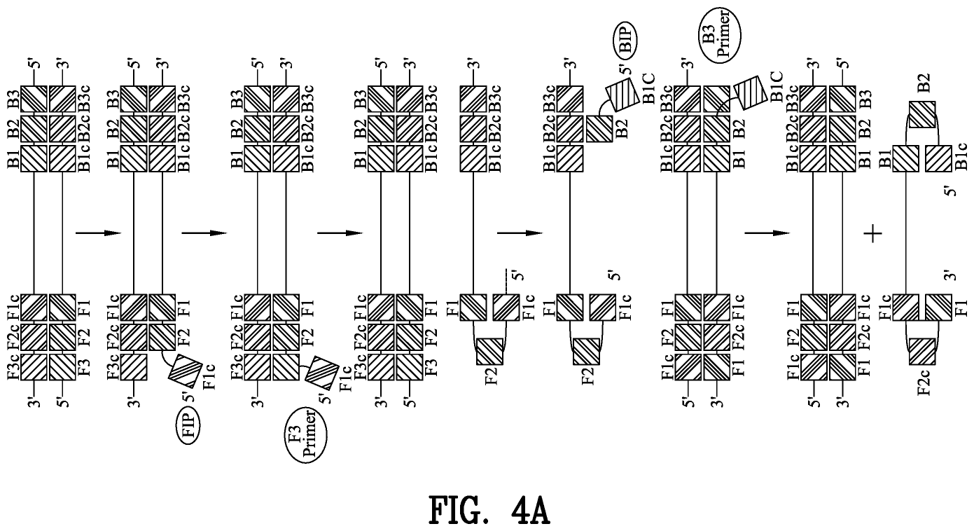

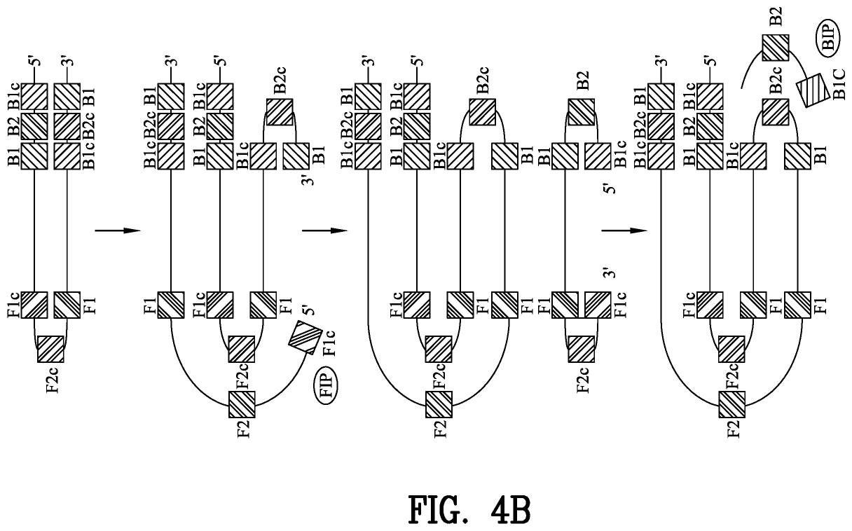

FIGS. 4A and 4B show the FIP primer having two regions of F2 which is complementary to F2c region of the template, and F1 c which is identical to the F1c sequence of the target. Similarly, BIP primer is consisted of B2 and B1c regions which are complementary to B2c and identical to Blc regions of the target sequence in order. FOP (also known as F3) and BOP (also known as B3) are complementary to the F3c and B3c regions of the template DNA.

### Stages of LAMP

Briefly, the F2 sequence of FIP primer is hybridized to F2c sequence of the template and initiates the amplification process. The F3 primer is then hybridized to F3c sequence of the template and starts the extension process. At the same time, F3 displaces the linked FIP strand and creates a single strand loop at the 5′ end of the extending. This looped-end single stranded DNA molecule operates as a target sequence for the BIP primer. At this point, B2 region is linked to B2c sequence of the target DNA and initiates the extension of the DNA molecule. The loop at the end of this template molecule is then opened at the end. The B3 primer is then hybridized to the B3c region of the molecule and displaces the linked BIP molecule and make a dumbbell shaped DNA molecule with two single stranded loops at each end. At this point, the DNA polymerase starts extending the DNA at the 3′ end of F1, opening the 5′ end-loop and forming a stem loop structure of the DNA. This structure performs as another template for LAMP. The FIP primer again hybridizes to the loop of the stem-loop DNA structure, initiates the extension of the DNA, displacing the F1 which leads to the formation of a new loop at the at the 3′ end. DNA polymerase then adds nucleotides to the 3′ end of B1, and displaces FIP strand, which leads to the formation of another dumbbell shaped DNA molecule. At this point, there will be a stem loop DNA and a gap repaired stem loop DNA, both of which serve as template other rounds of strand displacement reaction and elongation in the following cycles, which produce a mixture of stem looped-DNA with different stem lengths and several loops [2].

### Method of Operation

In this example, the rotary disk 1.0 contains 63 reaction chambers enabling 63 different tests to be simultaneously run from a single rotary disk 1.0. In the matter of 30 minutes, samples will be examined for 30 individual samples. However, it should be understood by those skilled in the art that the rotary disk 1.0 may be modified in design to accommodate higher or lower number of testing chambers.

An example of the method of operation of the apparatus is set forth below for a disk design with channels numbered 1 to 63 for each chamber. Chambers 1 to 3 will be used for negative and positive control tests. Chambers 5 and 6 will be used for the first patient and chambers 7 and 8 for the next patient and so on. Two chambers will be used for each sample, one for the detection of Covid-19 RNA, and one for the detection of the housekeeping gene as a proof of the accuracy of the DNA/RNA extraction prior to the test in the absence of Covid-19 genetic material.

Here is a non-limiting example of the injection protocol: First 1 to 9 μL of ddH₂O from Cartridge 3.1 will be injected in all 63 inlets. Then 12.5 μL of common biomaterial will be injected from cartridge 3.2 in all 63 inlets. Then from cartridge 3.3, 2.5 μL of housekeeping primer mix will be injected in inlets of chamber 2, 5, 7, 9, . . . , 63. Then from cartridge 3.4, 2.5 μL of N-gene and E-gene primer mix of Covid-19 will be injected in the inlet of chamber 3, 6, 8, 10, . . . , 62. The last step is to insert 1 μL of Covid-19 RNA from cartridge 3.6 into chamber 1, and 2. This step is called initialization and now disk is ready to accept clinical samples.

Sample 1 is injected into both chambers of 4 and 5. As explained above, chamber 4 has the human housekeeping gene primer, ddH₂O, common material whereas chamber 5 has common material, ddH₂O, N gene primer and E gene primer. If the device is tuned to do CRISPER or PCR, then the florescence emitting material can be engineered to have shift in the spectrum for both E and N genes.

The PCR process is conducted via thermal cycler, which is a laboratory apparatus capable of heating and cooling the samples in a holding block in multiple cycles to create the conditions necessary for the in vitro replication of the DNA molecule by DNA polymerase. Thermal cycling for PCR involves three main phases: 1) Denaturation (94° C. to 98° C.), in which double-stranded DNA templates are heated to separate the DNA strands; 2) Annealing (48° C. to 72° C.), in which primers bind to specific regions of the target DNA: and 3) Extension (68° C. to 72° C.), in which DNA polymerase extends the 3′ end of each primer based on the template strands. These steps are repeated in cycles to exponentially replicate the copies of the target DNA.

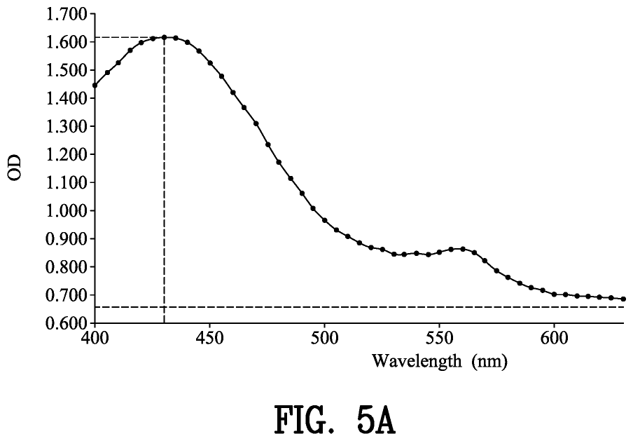

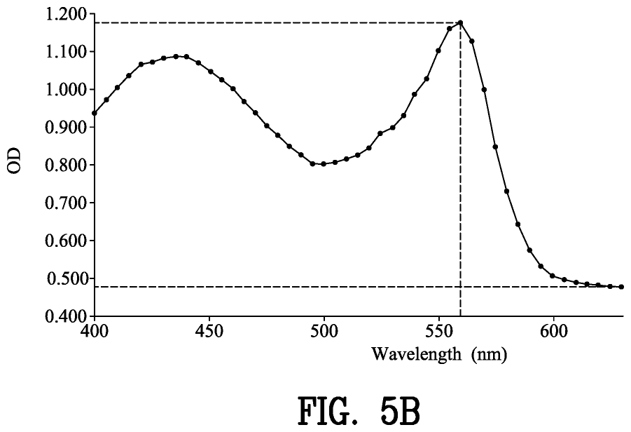

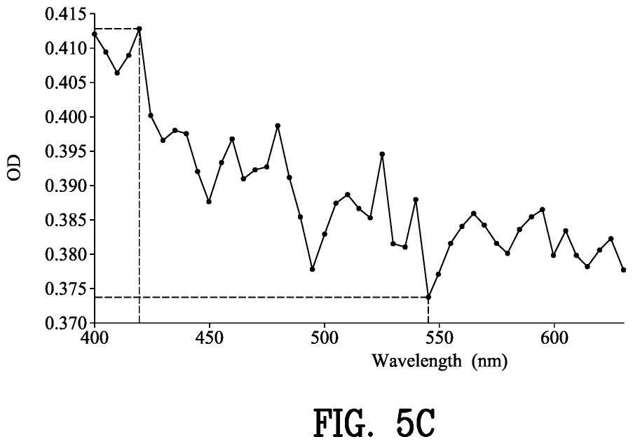

FIGS. 5A-C illustrate the absorption spectrum of the radiated light from chamber. The sample is irradiated with a light source and spectrometer detects the presence of the amplified DNA through absorption spectrum, indicating the presence of Covid-19 in the original sample. FIG. 5A presents the absorption spectrum of the sample, indicating yellow color. This is the positive control and sample contains Covid-19 genes. Before reaction starts the original color in the reaction chamber was violet-purple.

FIG. 5B presents the absorption spectrum of negative control. It was originally violet-purple and it stays violet-purple.

FIG. 5C presents the absorption spectrum from water only as reference signal.

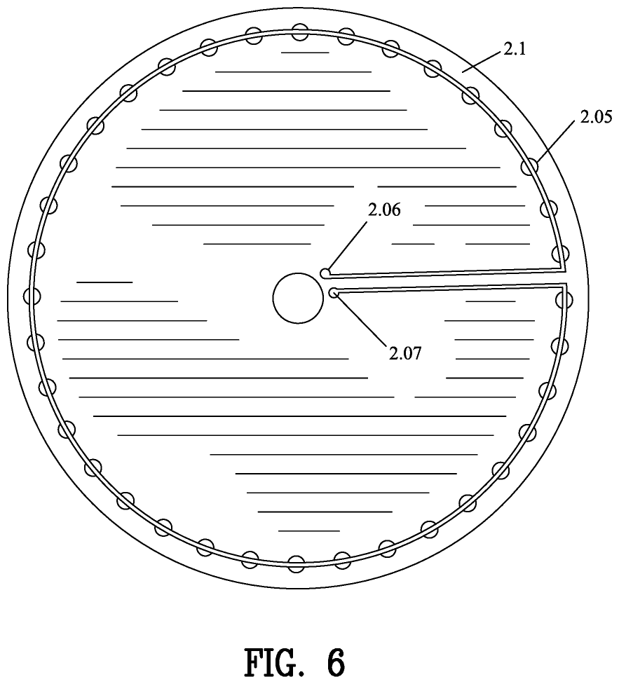

FIG. 6 presents another method of heating the sampling disk 1.0. In this method the base glass layer 2.1 is printed with transparent electrode such as indium Tin Oxide (ITO). The coating 2.05 is done only in the reaction chamber locations. By applying voltage at the electrodes 2.06 and 2.07 all the reaction chambers reach to 65° C. in 30 minutes. This type of local heating can be well controlled specially in the case that thermal cycling is needed.

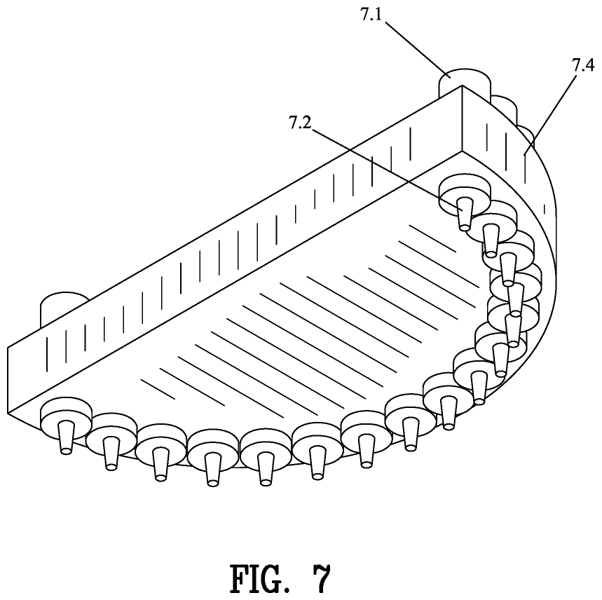

FIG. 7 is a schematic view of sample cartridge holder. In the FIG. 1 only the cross section is shown. The samples will be loaded in tubes 7.1. Each tube has barcode to assign for each individual sample. It has one needle 7.2. Entire holder 7.4 after filled up will be pushed in the machine to analyze.

The present disclosure includes that contained in the appended claims as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

What is claimed is:

1. An apparatus for detecting a microorganism within sampling device, the sampling device having a plurality of reaction chambers each having a reactive agent for reacting with the microorganism to indicate the presence of the microorganism in said reaction chamber, comprising:
a grabber for holding the sampling device;
a motion stage connected to said grabber for moving the sampling device in a plane; and
a detector for detecting each of the plurality of reaction chambers for detecting the presence of the microorganism in said reaction chamber.

2. The apparatus for detecting the Microorganism within sampling device as set forth in claim 1, wherein the sampling device is a rotatable disk having a central hole; and
said grabber engaging with the central hole for holding and rotating said rotary disk.

3. The apparatus for detecting the Microorganism within sampling device as set forth in claim 1, wherein said detector is a spectrometer for detecting the presence of the microorganism in the reaction chamber.

4. The apparatus for detecting the Microorganism within sampling device as set forth in claim 1, wherein said detector includes a light source located on one side of the sampling device and a spectrometer located on another side of the sampling device for detecting the presence of the microorganism in the reaction chamber.

5. The apparatus for detecting the Microorganism within sampling device as set forth in claim 1, wherein said detector includes a light source for irradiating the reaction chambers to indicate a reaction between the reactive agent and the microorganism; and
spectrometer for detecting a florescence radiation in the reaction chamber indicative of the presence of the microorganism in the reaction chamber.

6. The apparatus for detecting the Microorganism within sampling device as set forth in claim 1, including a cartridge having a plurality of containers for introducing reactive agents into the sampling device.

7. The apparatus for detecting the microorganism within sampling device as set forth in claim 1, including a cartridge having a plurality of containers for introducing samples into the sampling device.

8. A microfluidic sampling device for identifying the presence of a microorganism from a potential patient sample, comprising:
a sample body having an inlet for receiving the patient sample and reactive agents;
a reservoir for receiving the patient sample and a reactive agent from said inlet;
a microchannel for transferring the patient sample and said reactive agent from said reservoir to a reaction chamber;
said microchannel having a path for mixing the patient sample and said reactive agent prior to entering said reaction chamber; and
said reaction chamber being transparent for enabling said detection of the presence of the microorganism within said reaction chamber.

9. The microfluidic sampling device as set forth in claim 8, wherein said sample body is a rotary disk.

10. The microfluidic sampling device as set forth in claim 8, wherein said microchannel transfers the patient sample and said reactive agent from said reservoir to said reaction chamber upon rotation of said sample body.

11. The microfluidic sampling device as set forth in claim 8, wherein said microchannel has a restrictive path for creating an extra resistance for the patient sample and said reactive agent for mixing the patient sample with said reactive agent.

12. The microfluidic sampling device as set forth in claim 8, wherein said microchannel has a restrictive path including a siphon valve to create an extra resistance for the patient sample and said reactive agent for mixing the patient sample with said reactive agent.

13. The microfluidic sampling device as set forth in claim 8, wherein said channels connect to the reaction chamber by a siphon valve for backflow prevention.

14. The microfluidic sampling device as set forth in claim 8, wherein air evacuates from said reaction chamber through a vent channel.

15. A microfluidic sampling device for identifying the presence of Covid-19 from a patient sample, comprising:
a rotary disk having a plurality of inlets located on and inner region of said rotary disk for receiving potential patient samples;
a plurality of reservoirs for receiving the clinical sample and said reactive agent from said plurality of inlets;
a plurality of reaction chambers located on the outer periphery of said rotary disk;
a plurality of specific transparent patterned electrode on the bottom and cover layer for locally heating all reaction chambers at the same time;
a mixer channel for transferring the patient samples and said reactive agent from each of said plurality of reservoirs to each of said plurality of reaction chambers;
each of said microchannels having a restrictive path for mixing each of the patient sample with said reactive agents prior to entering each of said reaction chambers; and
said reaction chamber being transparent for enabling detection of the presence of Covid-19 within said reaction chambers.

16. The microfluidic sampling device for identifying the presence of Covid-19 as set forth in claim 15, wherein said rotary body is formed with glass or a polymeric material.

17. A method for rapid identifying the presence of Covid-19 from a potential patient sample, comprising the steps of:
introducing a reagent into a reservoir as preloaded;
introducing the patient sample into the reservoir;
shaking the patient sample and the reagent through back and forth rotation motion;
moving the mixed patient sample and reagent to a reaction chamber by a microchannel;
heating the patient sample and reagent within the reaction chamber; and
detecting the presence of Covid-19 within the reaction chamber.

18. A method for rapid identifying the presence of Covid-19 as set forth in claim 17, wherein the step of moving the patient sample and the reagent includes rotating the reservoir for creating a centrifugal force.

19. A method for rapid identifying the presence of a microorganism from a potential patient sample, comprising the steps of:
introducing the patient sample into a reservoir;
introducing the reagent into the reservoir;
shaking the patient sample and the reagent by back and forth motion to mix the patient sample with the reagent;
moving the patient sample and the reagent to a reaction chamber;
thermal cycling the patient sample and the reagent within the reaction chamber for polymerize chain reaction (PCR); and
detecting the presence of the infectious microorganisms within the reaction chamber.

20. The method for rapid identifying the presence of a microorganism as set forth in claim 19, wherein the step of moving the patient sample and the reagent includes rotating the reservoir for creating a centrifugal force.
